(19) United States
(12) Patent Application Publication
(10) Pub. No.: US 2004/0253305 A1
(43) Pub. Date: Dec. 16, 2004

(54) Pharmaceutical compositions of atorvastatin

(76) Inventors: Paul E. Luner, Waterford, CT (US); Kenneth Craig Waterman, East Lyme, CT (US)

Correspondence Address: WARNER-LAMBERT COMPANY, 2800 PLYMOUTH RD, ANN ARBOR, MI, 48105, US

(21) Appl. No.: 10/828,398

(22) Filed: Apr. 20, 2004

Related U.S. Application Data
(60) Provisional application No. 60/477,916, filed on Jun. 12, 2003.
(60) Provisional application No. 60/477,917, filed on Jun. 12, 2003.

(51) Int. Cl. A61K 31/401; A61K 9/48; A61K 9/20

(52) U.S. Cl. 424/451; 514/423; 424/464

(57) ABSTRACT

A dry-granulated pharmaceutical composition comprising atorvastatin or a pharmaceutically acceptable salt thereof, as well as a dry-granulated pharmaceutical composition comprising atorvastatin or a pharmaceutically acceptable salt thereof in combination with at least one other active drug, methods for preparing said compositions, kits for containing such compositions, and a method of treating hypercholesterolemia and/or hyperlipidemia, osteoporosis, benign prostatic hyperplasia (BPH), and Alzheimer's disease using a therapeutically effective amount of the pharmaceutical composition.

### CROSS-REFERENCE TO RELATED APPLICATIONS

This application claims priority from U.S. Provisional Patent Application No. 60/477,916 filed Jun. 12, 2003.

### FIELD OF THE INVENTION

This invention relates to pharmaceutical compositions comprising atorvastatin and pharmaceutically acceptable salts thereof and a process for the preparation of the same, kits containing such compositions, as well as methods of using such compositions to treat subjects suffering from hypercholesterolemia and/or hyperlipidemia, as well as osteoporosis, benign prostatic hyperplasia (BPH), and Alzheimer's disease.

### BACKGROUND OF THE INVENTION

The conversion of 3-hydroxy-3-methylglutaryl-coenzyme A (HMG-CoA) to mevalonate is an early and rate-limiting step in the cholesterol biosynthetic pathway. This step is catalyzed by the enzyme HMG-CoA reductase. Statins inhibit HMG-CoA reductase from catalyzing this conversion. As such, statins are collectively potent lipid lowering agents.

Atorvastatin calcium, disclosed in U.S. Pat. No. 5,273,995 which is incorporated herein by reference, is currently sold as Lipitor® having the chemical name [R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid calcium salt (2:1) trihydrate and the formula

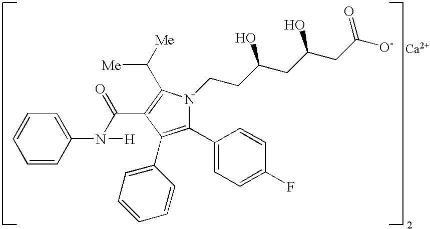

Atorvastatin and pharmaceutically acceptable salts thereof are selective, competitive inhibitors of HMG-CoA reductase. As such, atorvastatin calcium is a potent lipid-lowering compound and is thus useful as a hypolipidemic and/or hypocholesterolemic agent, as well as in the treatment of osteoporosis, benign prostatic hyperplasia (BPH), and Alzheimer's disease.

A number of patents have issued disclosing atorvastatin, formulations of atorvastatin, as well as processes and key intermediates for preparing atorvastatin. These include: U.S. Pat. Nos. 4,681,893; 5,273,995; 5,003,080, 5,097,045; 5,103,024; 5,124,482; 5,149,837; 5,155,251; 5,216,174; 5,245,047; 5,248,793; 5,280,126; 5,397,792; 5,342,952; 5,298,627; 5,446,054; 5,470,981; 5,489,690; 5,489,691; 5,510,488; 5,686,104; 5,998,633; 6,087,511; 6,126,971; 6,433,213; and 6,476,235, which are herein incorporated by reference.

Atorvastatin can exist in crystalline, liquid crystalline and non-crystalline and amorphous forms.

Crystalline forms of atorvastatin calcium are disclosed in U.S. Pat. Nos. 5,969,156 and 6,121,461, which are herein incorporated by reference. Further crystalline forms of atorvastatin are disclosed U.S. Pat. No. 6,605,729 which is herein incorporated by reference.

Additionally, a number of published International Patent Applications have disclosed crystalline forms of atorvastatin, as well as processes for preparing amorphous atorvastatin. These include: WO 00/71116; WO 01/28999; WO 01/36384; WO 01/42209; WO 02/41834; WO 02/43667; WO 02/43732; WO 02/051804; WO 02/057228; WO 02/057229; WO 02/057274; WO 02/059087; WO 02/083637; WO 02/083638; WO 03/011826; WO 03/050085; WO 03/070702; and WO 04/022053.

It has been disclosed that the amorphous forms in a number of drugs exhibit different dissolution characteristics and in some cases different bioavailability patterns compared to the crystalline form (Konno, T., Chem. Pharm. Bull., 1990;38:2003-2007). For some therapeutic indications one bioavailability pattern may be favored over another.

Variations in dissolution rates can make it advantageous to produce atorvastatin formulations in either crystalline or amorphous forms. For example, for some potential uses of atorvastatin (e.g., acute treatment of patients having strokes as described in Takemoto, M.; Node, K.; Nakagami, H.; Liao, Y.; Grimm, M.; Takemoto, Y.; Kitakaze, M.; Liao, J. K., Journal of Clinical Investigation, 2001; 108(10): 1429-1437) a rapid onset of activity may be highly beneficial in improving the efficacy of the drug.

The preparation of solid formulations of atorvastatin is described in U.S. Pat. Nos. 5,686,104 and 6,126,971. In the process described therein, atorvastatin is combined with a stabilizing additive, such as, an alkaline earth metal salt and excipients and subjected to wet granulation using a combination of water and a surfactant (Tween 80). Because alkaline earth metal salts can at times affect atorvastatin bioavailability, there remains a need to provide atorvastatin in a formulation that minimizes the level of alkaline earth metal salts.

In preparation and storage of unit dosage forms of atorvastatin, it is important to provide the active drug in a pure form. Moreover, it is desirable to achieve this high purity and stability with as simple a formulation as possible. There remains a need to provide simple formulations and processes for preparation of unit dosage forms of atorvastatin which have low levels of impurities and provide adequate stability to allow dosage form expiration times that are commercially viable.

Since atorvastatin is a highly potent drug, formulations of the drug are generally quite dilute in order to provide dosage forms of adequate size for manufacturing and ease of handling by patients. When a drug is used in a dilute form, the risk exists that segregation between the drug and excipients during the processes before the drug is in its final dosage form could lead to some of the unit dosage forms being hypo or hyperpotent. Potency control of the unit dosage forms is essential to prevent individual patients from receiving an incorrect, and sub-therapeutic or side effect generating dose of the drug. Granulations are one method for preventing segregation. Although it is possible to select excipients such that unit dosage forms can be prepared without a granulation step, as disclosed in concurrently filed United States patent application, commonly owned, attorney case number PC25684, Ser. No. ______, granulations can assure that drug and excipients are bound together such that segregation will not occur and the particle size of the granules will allow for good flow. Wet granulations represent one option for providing atorvastatin in a form unlikely to segregate and with good flow (see concurrently filed United States patent application, commonly owned, attorney case number PC25685, Ser. No. ______). Wet granulations, however, require the formulation to be exposed to water and/or solvents. Such exposure increases the risk that the solid-state form of the atorvastatin could change (e.g., crystallize or change polymorphic form) or degrade chemically. Since liquid addition amount and rate will depend on such factors as the volume and surface area of the wet granulation vessels and on the exact particle sizes of the drug and excipients used in a specific manufacturing run, there can be difficulties in scaling-up wet granulation processes (i.e., variability in performance). It is therefore the purpose of the present invention to provide dry granulation formulations and processes for atorvastatin such that drug segregation is minimized, flow of said composition is acceptable for commercial unit dosage formation, drug will not be exposed to a solvent and a robust (scalable) process is employed.

In dry granulation processes, typically the drug and at least some of the excipients are pressed together to form ribbons or slugs. These compacted materials are then milled to an appropriate size to prevent drug segregation and assure good flow during the production of unit dosage forms. We have found that while the drug itself will compress to form slugs, upon milling, the material reverts predominantly back to a fine powder with poor flowing properties. There remains a need therefore to provide compositions suitable for dry granulation of atorvastatin that provide adequate flow of the drug such that unit dosage forms can be prepared with good weight control.

It is an object of the present invention to provide compositions and processes for producing dosage forms of atorvastatin having good dose-to-dose potency uniformity, dissolution rates and bioavailability. It is a further object of the present invention to provide a stable and pure composition of atorvastatin, in crystalline or amorphous form, with minimal addition of alkaline metal salts.

### SUMMARY OF THE INVENTION

A first aspect of the present invention is a dry-granulated pharmaceutical composition comprising atorvastatin or a pharmaceutically acceptable salt thereof.

A second aspect of the present invention is a method for preparing a dry-granulated pharmaceutical composition of atorvastatin comprising:

(a) combining atorvastatin or a pharmaceutically acceptable salt thereof and one or more excipients suitable for use in a dry granulation step;

(b) blending the mixture together in a mixer;

(c) compressing the mixture;

(d) milling, grinding or sieving the compressed material;

(e) optionally adding additional excipients and mixing the combination to form the composition.

A third aspect of the present invention is a dry-granulated pharmaceutical composition comprising atorvastatin or a pharmaceutically acceptable salt thereof in combination with at least one other active drug.

A fourth aspect of the present invention is a method for preparing a dry-granulated pharmaceutical composition of atorvastatin comprising:

(a) combining atorvastatin or a pharmaceutically acceptable salt thereof in combination with at least one active drug and one or more excipients suitable for use in a dry granulation step;

(b) blending the mixture together in a mixer;

(c) compressing the mixture;

(d) milling, grinding or sieving the compressed material;

(e) optionally adding additional excipients and mixing the combination to form the composition.

A fifth aspect of the present invention is a therapeutic package or kit suitable for commercial sale, comprising a container and a therapeutically effective amount of dry-granulated atorvastatin or a pharmaceutically acceptable salt thereof.

A sixth aspect of the present invention is a method of using a dry-granulated atoravastatin composition to treat subjects suffering from hypercholesterolemia and/or hyperlipidemia, osteoporosis, benign prostatic hyperplasia (BPH), and Alzheimer's disease.

### DETAILED DESCRIPTION OF THE INVENTION

Atorvastatin can readily be prepared as described in U.S. Pat. Nos. 4,681,893, 5,273,995 and 5,969,156, which are incorporated herein by reference. The hemicalcium salt of atorvastatin is currently sold as Lipitor®.

Atorvastatin exists in a number of morphological forms ranging from highly crystalline forms to forms with varying degrees of disorder. Some of these disordered forms still possess some structure as indicated by powder x-ray diffraction patterns. For the purpose of the present invention, all forms of atorvastatin benefit from the invention and are included within the scope of the invention. Partially or completely disordered forms of atorvastatin particularly benefit from the invention. Partially or completely disordered forms of atorvastatin that are amorphous or predominantly amorphous derive the greatest benefit from the present invention. Such forms can be prepared, for example, from crystalline atorvastatin using procedures disclosed in U.S. Pat. No. 6,087,511, which is incorporated herein by reference. Alternatively, amorphous material can be prepared according to the processes disclosed in United States patent application, commonly owned, attorney's case number PC-25825 Ser. No. ______. For the practice of the present invention, non-crystalline and crystalline atorvastatin can be prepared by any method known in the art. Preferred forms of atorvastatin are described in U.S. Pat. Nos. 5,969,156, 6,121,461, and 6,605,729; and in International Patent Applications WO 01/36384, WO 02/41834; WO 02/43732; WO 02/051804, WO 02/057228, WO 02/057229, WO 03/011826, WO 03/050085, WO 03/070702, and WO 04/022053, which are incorporated herein by reference.

The atorvastatin can be used in the form in which it is prepared, or it can be subjected to a process which changes the physical nature of the particles. For example, the material can be milled by any process known in the art. Non-exclusive examples of such processes include mechanical milling and jet milling. The particles produced either directly from the process of forming atorvastatin or after a milling operation preferably provide mean particle diameters in the range of 1-200 μm; more preferably between 5 and 150 μm.

Pharmaceutically acceptable base addition salts of atorvastatin are formed with metals or amines, such as alkaline and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, S. M., et al., “Pharmaceutical Salts”, J. Pharm. Sci., 1977; 66:1).

The base addition salts of atorvastatin are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. Additionally, atorvastatin can exist in unsolvated forms as well as solvated forms, including hydrated forms. In general, the solvated forms, including hydrated forms, are intended to be encompassed within the scope of the present invention.

Forms of atorvastatin that are at least somewhat disordered or a mixture of crystalline and disordered forms of atorvastatin benefit most significantly from the present invention. By somewhat disordered, it is meant that the line width (peak width at half the height of the peak) of any of the peaks measured using powder x-ray diffraction (PXRD) have 2 theta values greater than about 2°. Amorphous or predominantly amorphous forms of atorvastatin, which especially benefit from the present invention, are characterized by having very broad, featureless peaks. It should be noted that combinations of crystalline and at least somewhat disordered forms of atorvastatin will show both sharp (i.e., less than 2° values for 2 theta) and broad peaks (i.e., greater than 2°), and such combinations of forms benefit from the present invention.

Atorvastatin has been found to be an effective drug even at relatively low doses. In fact, by keeping the dose low for a given patient, it is possible to minimize side-effects while still maintaining drug efficacy. It is therefore desirable to provide atorvastatin in a form capable of providing a low dose to the patient. For the purposes of the present invention, the dose provided by the final dosage form of atorvastatin is preferably between 0.5 and 120 mgA (where mgA means milligrams of active drug based on the free acid); more preferably, between 5 and 80 mgA.

For convenience and ease of patient compliance, most drugs are delivered in the form of unit dosage forms. For solid drug substances, these unit dosage forms are generally in the form of tablets, capsules, sachets, chewable tablets and fast dissolving dosage forms. In the present invention, the dosage form is preferably in the form of a capsule or tablet; most preferably in the form of a tablet. The preparation of these forms involves a necessary step of some type of powder filling, either by volume or weight. For example, in production of tablets and capsules, powder is volume filled into a die or capsule, respectively. In order for the unit dosage forms to have the same potency (i.e., amount of drug per unit dosage form) within allowable margins (relative standard deviation, RSD, of less than 6% to meet Stage I, and less than 7.8% to meet Stage II of the United States Pharmacopoeia, USP, guidelines), there must not be any significant segregation of the active drug from the excipients. This is especially significant for highly dilute forms. The present invention discloses compositions that provide reproducible potency for a fixed weight of active atorvastatin plus excipients. Moreover, this potency control is maintained through the process of producing unit dosage forms. Such compositions, before being processed into unit dosage forms, provide atorvastatin with potency (mgA per gram of blend) variability of less than an RSD of 7.8%; more preferably, less than 6.0%. In addition, the present compositions provide for good powder flow such that weight control is maintained between unit dosage forms produced with such compositions (i.e., variability in the weights of unit dosage forms produced from such compositions is minimal). Preferably, such compositions provide unit dosage forms with weight control within an RSD of 6%; more preferably, within 5%; even more preferably, within 4%. Combining the weight control and the potency control allows the present compositions to provide unit dosage forms with potencies of atorvastatin per unit dosage form having an RSD preferably less than 7.8%; more preferably less than 6.0%.

Measurement of the potency of unit dosage forms of atorvastatin is necessary in determining the variability in activity between unit dosage forms. An extraction process against a standard with independently known drug levels best determines such potency. The potency analysis is best conducted using reverse phase high performance liquid chromatography (HPLC) techniques such as those known in the art relative to standards. RSD measurements, for the purpose of the present invention, are best carried out using sampling during a process for forming the unit dosage form. More specifically, unit dosage forms can be sampled from a preparation process at various time points (beginning, middle and end of the run). In determining an RSD value, at least three unit dosage forms should be measured from each section. An alternative analytical technique for determining the potency of a sample of drug involves the use of ultraviolet-visible absorption spectroscopy. In this technique, the absorbance corresponding to atorvastatin is used to quantify the concentration of atorvastatin in a sample (taking care that no excipient has interfering absorptions), as is known in the art.

The present invention discloses processes and compositions that provide atorvastatin in a pure and stable form. The term “impurities” describes materials in the drug substance present from the synthesis and purification process and any drug-based materials formed in the preparation of the unit dosage form. The term “degradants” refers to any drug-based materials generated after the preparation of the unit dosage form. Analysis of impurities and degradants is done using reverse phase HPLC techniques on extracted samples as is known in the art. Calculations of the amount of impurities and degradants is expressed as the integrated area percent of the degradant or impurity peak(s) divided by the integrated area percent of all drug-related peaks.

The particle sizes of the atorvastatin and the excipients play a significant role in the effectiveness of the dry granulation process in preventing segregation. As such, mean particle sizes can be measured using a laser diffraction particle size instrument such as those made by Sympatec GmbH (Goslar, Germany). Mean particle sizes, for the purpose of the present invention, can be considered the size for which 50% of the particles have diameters smaller than the indicated number. Alternatively, particle size can be assessed using sieve analysis. The percent of the total weight of material retained on sieves of particular sizes is used to measure the mean particle size. The mean particle size is the sieve size that allows about 50% of the weight of material to pass through (50% retained).

In the preparation of compositons of atorvastatin with a dry granulation, combinations of diluents, binders, disintegrants, flavorants and lubricants are used to provide the properties needed for the unit dosage form as is known in the art. For example, for preparation of tablets, the combination provides for adequate tablet hardness upon compression while providing rapid disintegration in vivo. Although there is a wide degree of latitude in formulating atorvastatin to meet these conditions, typically such formulations contain about 140% (w:w) drug, about 5-10% disintegrant, about 0-10% binder and about 0.5-2% lubricant, with the remaining percentage comprising the inventive diluents. Preferred disintegrants include carboxymethylcellulose, hydroxyproyl cellulose (low-substituted), microcrystalline cellulose, powdered cellulose, colloidal silicon dioxide, croscarmellose sodium, crospovidone, magnesium aluminum silicate, methylcellulose, polacrilin potassium, povidone, sodium alginate, sodium starch glycolate and starches. Preferred binders include acacia, carboxymethylcellulose, hydroxyethylcellulose, hydroxypropylcellulose, dextrin, gelatin, guar gum, hydroxypropyl methylcellulose, magnesium aluminum silicate, maltodextrin, methylcellulose, polyethylene oxide, polymethacrylates, povidone, sodium alginate, starches and zein. Preferred lubricants include calcium stearate, glyceryl palmitostearate, magnesium oxide, poloxamer, polyethylene glycol, polyvinyl alcohol, sodium benzoate, sodium lauryl sulfate, sodium stearyl fumarate, stearic acid, talc, zinc stearate and magnesium stearate.

In improving the flow of atorvastatin compositions and minimizing segregation from excipients, compaction of the drug with excipients can be carried out in a dry granulation process. With atorvastatin, however, we have found that due to the brittle nature of the drug, only certain excipients form acceptable dry granulations with the drug. Acceptable excipients can be defined in terms of those which when dry granulated (i.e., compacted and milled) in the presence of atorvastatin, provide for a significant reduction in the amount of fine drug particles, unbound to excipient, remaining in the blend. For the purpose of the present invention, fine drug particles (or “fines”) can be defined as particles that pass through a 200 mesh sieve. We can also define a granulation factor as: granulation factor (GF)=1−[(percentage atorvastatin as fines in granulation)/(percentage of atorvastatin as fines in ungranulated blend)]. To determine the granulation factor for a given excipient or excipient combination, one first prepares a blend of the drug with the selected excipients. This blend is passed through sieves using a sifting device such as a Sonic Sifter™ (Allen Bradley Sonic Sifter, Advantech Manufacturing, New Berlin, Wis.). The percentage atorvastatin as fines is determined by multiplying the weight of by the potency of the fines divided by the overall weight of drug in the blend. The same analysis can be conducted on material that has been dry granulated with atorvastatin. From these analyses, the overall ability of a given excipient or set of excipients to form formulations with a low tendency to segregate can be determined. We have found that excipient or excipient combinations with atorvastatin that provide for minimal tendency to segregate during unit dosage form preparation can be characterized as having granulation factors preferably between 0.4 and 1.0; more preferably, between 0.5 and 1.0; and still more preferably between 0.6 and 1.0.

We have found that in determining suitable excipients for dry granulation with atorvastatin, compression of blends under conditions translatable to commercial dry granulation is important to determining the true tendency for atorvastatin blends to segregate. One of the important criteria to consider when granulating atorvastatin is the solid fraction of compacts or ribbons of the blend, especially in cases where the granulation undergoes an additional compression step such as occurs during formation of tablets. The solid fraction is an indication of the amount of compression remaining in the material. As such, the first step involves determining the true density of the blend, i.e., the density of the materials without air spaces between particles. This density can be measured using such techniques as helium pycnometry or similar techniques, as is known in the art. It is also possible to estimate this value as a weighted average of the true density values for each of the components. The solid fraction of a dry granulation represents the ratio of the density of a compact (or ribbon) to the true density of the material from which the compact was made. Control of the solid fraction is achieved by controlling the compression forces during compaction. We have found that to achieve good binding of atorvastatin while still providing sufficient compressibility for subsequent tableting, granulations preferably have a solid fraction after granulation between about 0.55 and 0.85; more preferably between about 0.60 and 0.80.

Another criterion for achieving acceptable dry granulations of atorvastatin is the tensile strength of the compacts or ribbons. The tensile strength of a compact (or ribbon) can be measured using appropriate equipment as is known in the art, such as, a CT5 tensile strength Tester (Engineering System (NOTTM), Nottingham, England). Preferably, rectangular compacts having dimensions of 10×22×2 mm are used for this measurement. We have found that a preferred tensile strength for ultimately producing acceptable granulations of atorvastatin is 0.5 to 7.0 megapascals (Mpa); more preferably, 0.8 to 6.0 MPa. Combinations of materials with atorvastatin that can achieve the preferred tensile strength within the range of preferred solid fractions are preferred. Examples of such materials include lactose and microcrystalline cellulose. An example of a material unable to achieve the desired tensile strength is mannitol.

By plotting the solid fraction versus the tensile strength, it is possible to find a solid fraction range appropriate for providing the preferred tensile strength with a given blend of atorvastatin and an excipient or excipient combination. We have found that interpolation between measured values can be used, assuming an exponential least squares fitting.

Preferred excipients are diluents, which preferably comprise greater than or equal to 40 wt % of the total composition in the formulation with atorvastatin; more preferably, greater than 50 wt %; still more preferably, greater than 60 wt %. Preferred diluents, when tested in binary blends with atorvastatin, provide granulation factors of preferably between 0.4 and 1.0; more preferably, between 0.5 and 1.0; and still more preferably between 0.6 and 1.0. Potential diluents are identified as such in “Handbook of Pharmaceutical Excipients, 3rd Edition” (A. H. Kibbe, Editor; Pharmaceutical Press, London; 2000). These include the following non-limiting examples: calcium phosphate, calcium sulfate, carboxymethylcellulose calcium, cellulose, cellulose acetate, dextrates, dextrin, dextrose, fructose, glyceryl palmitostearate, hydrogenated vegetable oil, kaolin, lactitol, lactose, magnesium carbonate, magnesium oxide, maltitol, maltodextrin, maltose, polymethacrylates, pregelatinized starch, silicified microcrystalline cellulose, sodium chloride, sorbitol, starch, sucrose and talc.

In determining excipients appropriate for use in dry granulations with atorvastatin, it is important that the particular form and particle size of atorvastatin be used that is desired for the final dosage form. Similarly, the excipient or excipient combination used will have properties that depend on the particle size and method of preparation. Since compression of the excipients with atorvastatin in a dry granulation process is generally more facile with smaller particle-size excipients, preferred excipients are generally smaller than would be preferred without granulation. As such, preferably the excipients have mean particle sizes between 20 and 200 μm; more preferably, between 40 and 150 μm. These particle size ranges correspond to 50 weight % of the blend passing through sieves having between a 635 mesh sieve (ASTM number) and a 70 mesh; more preferably, between a 325 mesh and a 100 mesh. The preferred size for a given excipient depends on the specific properties of the atorvastatin form used and must be determined experimentally in each case.

More preferred excipients in combination with atorvastatin, therefore, are diluents that provide high values of granulation factors, can achieve a high tensile strength and have preferred mean particle sizes, preferably between 20 and 200 μm; more preferably, between 40 and 150 μm. Particularly preferred diluents include microcrystalline celluloses having a mean particles size of 20 to 40 μm (such as Avicel™ PH105, available from FMC Biopolymer, Philadelphia, Pa.), lactoses having a particle size range of 80 to 150 μm (such as the spray dried monohydrate material or Fast Flo™ 316 available from Foremost Farms, Rothschild, Wis.; or the anhydrous, direct tableting grade, available from Quest International, Flavors & Food Ingredients CCL, Norwich, N.Y.), xylitol (such as the C granular grade available from Danisco Sweeteners, Thomson, Ill.), mannitol (such as Mannogem™ 2080 granular, available from SPI Polyols, New Castle, Del.), sucrose (such as Di-Pac™, available from Tate & Lyle Co. American Sugars Inc, Brooklyn, N.Y.), and calcium phosphate dibasic anhydrous (such as A-Tab™, available from Rhodia, Chicago Heights, Ill.). Preferably, the preferred diluents comprise greater than 50% (w:w) of the diluent content of the dry-granulated composition of atorvastatin; more preferably 60% (w:w); still more preferably 70% (w:w).

Unit dosage forms of atorvastatin that are formed with a dry granulation step with preferred excipients show low levels of drug-related impurities and degradants. Surprisingly, this low level of impurities and degradants was found even in the absence of added alkalizing agents or alkaline earth metal salts. Even more surprisingly, this low level of impurities and degradants was maintained even when the atorvastatin used was an at least somewhat disordered form of the drug. In particular, it was found that while wet granulated control unit dosage forms of atorvastatin show high levels of drug degradation, unit dosage forms prepared with dry granulation have greater stability. Those unit dosage forms of atorvastatin prepared with dry granulation are preferred that contain not more than about 2% total drug related impurities and/or degradants based on the area percent of the impurities/degradants relative to the integrated area of all drug related peaks as determined by HPLC; more preferably, they contain less than 1%; still more preferably, less than 0.7%. In addition, unit dosage forms of atorvastatin prepared with dry granulation are preferred that provide stability such that upon storage at 40° C. and 75% relative humidity (RH) for four weeks, the unit dosage forms contain not more than about 2% total drug related impurities and/or degradants based on the area percent of the impurities/degradants relative to the integrated area of all drug related peaks as determined by HPLC; more preferably, they contain less than 1%; still more preferably, less than 0.7%.

Atorvastatin undergoes two major degradation pathways: lactonization and oxidation. The lactone is formed by internal condensation (loss of water) of the alcohol and carboxylic acid to form a six-membered ring. This is the major degradant of amorphous atorvastatin found upon wet granulation and tablet formation as described in U.S. Pat. Nos. 6,126,971 and 5,686,104, especially in the absence of alkaline earth metal salts. We have found, unexpectedly, that the level of the lactone in unit dosage forms, both initially and upon storage under accelerated aging conditions of increased temperature and humidity, can be significantly reduced by combination of the present excipients and production of unit dosage forms using a dry granulation process. Preferably, the level of atorvastatin lactone in unit dosage forms is less than 2% (based on the ratio of lactone peak integration compared to the total peak integrated areas using HPLC) after said unit dosage forms are produced and stored at 40° C./75% RH (where RH represents relative humidity) for four weeks; more preferably, less than 1%.

To minimize bioavailability issues and potential interactions with other drugs in combination dosage forms, in the practice of the present invention, the level of alkaline earth metal salts in the formulation is preferably about 0-5% (w:w); more preferably, about 0-3%; most preferably about 0-2%. It is also preferred that the level of other alkalizing agents in the formulation be about 0-5% (w:w); more preferably, about 0-3%; most preferably about 0-2%.

Dry granulation of atorvastatin with excipients is preferably carried out by first blending the atorvastatin with at least some of the preferred excipients. Preferably, the excipients in this blend constitute between 50 and 95% (w:w) of the blend. This blending process is preferably carried out using a high shear mixer, V-blender (or other twin-shell blender), bin blender or Turbula™ mixer-shaker (available from Willy A. Bachofen AG Maschinenfabrik, Basel, Switzerland). Blending is typically carried out first without the addition of a lubricant for sufficient time to assure complete mixing. At that point, the lubricant is typically added followed by a short (about 1-10 minute) further mixing period. This blend is then compressed into slugs or ribbons using a tablet press (such as a single-station press or a rotary tablet press) or a roller compactor. In the former case, compacts (slugs) are produced using flat-faced die and punch combinations. In both cases, the density of the compacts or ribbons is preferably chosen to provide compacts or ribbons having tensile strengths of about 0.5 to 7.0 MPa; more preferably, about 0.8 to 6.0 MPa. The compacts or ribbons are then preferably milled, ground or sieved. The particle size reduction is carried out in optimized processes designed to give good throughput while providing a suitable particle size distribution, as is known in the art. Preferably, less than 30% (w:w) of the milled material will pass through a 200-mesh sieve and greater than 70% (w:w) will pass through a 60-mesh sieve. Once the material is milled, other excipients can be added extragranularly to provide the final blend for unit dosage form manufacturing. These additives are preferably mixed using a high shear mixer, V-blender (or other twin-shell blender), bin blender or Turbula™ mixer-shaker. Blending is typically carried out first without the addition of a lubricant for sufficient time to assure complete mixing. At that point, the lubricant is typically added followed by a short (about 1-10 minute) further mixing period. At this point, the granulated material can be used in the preparation of unit dosage forms. Such unit dosage forms include sachets, tablets, fast-dissolving dosage forms, chewable dosage forms and capsules. Preferred dosage forms include tablets and capsules. In the case of tablets, it can be desirable to coat them with a film designed to provide ease of swallowing, a proprietary or identification appearance and/or protection of the dosage form. The final dosage form is then packaged using procedures known in the art. For the present invention, the packaging is preferably in the form of foil-foil cold form blisters, plastic blisters or sealed bottles containing desiccants. Optionally, the packaging can contain active oxygen absorbing materials as is disclosed in EP1243524A2, which is incorporated herein by reference.

In production of unit dosage forms of atorvastatin with dry granulation, it is possible to produce such unit dosage forms without the present formulations using processes unsuitable to commercial production. For example, even granulated material with a tendency to segregate could be weighed into a capsule directly. The present invention, therefore, is preferably used in conjunction with high-speed production equipment. More specifically, preferred formulations provide dry granulations that allow potency control during unit dosage form production of less than 7.8% RSD (more preferably less than 6.0% RSD) when used with a single apparatus-unit dosage form production equipment at a rate of greater than 10,000 unit dosage forms per hour; more preferably, greater than 25,000 unit dosage forms per hour; most preferably, greater than 50,000 unit dosage forms per hour. Preferred single apparatus-unit dosage form production equipment or machines include single rotary tablet presses and a single commercial capsule filling machines. Non-exclusive examples of commercial rotary tablet presses include those produced by Niro Pharma Systems (Columbia, Md.), Kilian and Company (Horsham, Pa.), Korsch (Berline, Germany) and Elizabet-Hata International (North Huntingdon, Pa.). Non-exclusive examples of commercial capsule filling equipment include those made by Capsugel (Morris Plains, N.J.) and CapPlus Technologies (Phoenix, Ariz.).

The present invention provides for compositions of atorvastatin which are particularly well suited for combination products with other drug substances because the granulation does not require a potentially solubilizing and/or otherwise destabilizing solvent and yet maintains the atorvastatin content uniformity. This is especially true when the second drug (with its associated excipients) can destabilize atorvastatin. Non-limiting examples of drugs which may benefit from combinations with the inventive atorvastatin compositions and processes include torcetrapib and amlodipine and pharmaceutically acceptable salts thereof.

Compositions of atorvastatin according to the present invention can be combined with a least one other active drug to form unit dosage forms. Preferred unit dosage forms include tablets and capsules. In the combination of the atorvastatin composition with at least one other active drug to form a unit dosage form, the following non-limiting list describes options for such unit dosage forms: (a) a blend of the atorvastatin granulation with the other active drug itself (i.e., extragranular addition of the other drug to the dry granulated composition of atorvastatin), as a blend with excipients (i.e., extragranular addition of the other drug plus excipients to the dry granulated composition of atorvastatin), or as a granulation (i.e., a mixture of a granulation of the other drug with the dry granulated composition of atorvastatin), formed into tablets or capsules; (b) a single dry granulation of atorvastatin with the other drug, formed into tablets of capsules; (c) a bilayer tablet comprising dry granulated atorvastatin in one layer and the other drug and optional excipients in the other layer.

The present invention relates to the treatment of diseases and conditions in a subject, such as, hyperlipidemia and/or hypercholesterolemia, osteoporosis, benign prostatic hyperplasia (BPH), and Alzheimer's disease with atorvastatin or a pharmaceutically acceptable salt thereof as described above that may be administered in a unit dosage form having low levels of degradation products and/or impurities contained in a therapeutic package or kit. The kit includes the unit dosage form and a container. Typically, the kit includes directions for administration of the unit dosage form. The container can be in any conventional shape or form as known in the art, for example, a paper box, a glass or plastic bottle, or a blister pack with individual dosage forms pressing out of the back according to a therapeutic schedule.

The following non-limiting examples illustrate the inventors' preferred methods for preparing and using the pharmaceutical compositions of the present invention.

### EXAMPLE 1

### General Method for Preparation of Spray-Dried Amorphous Atorvastatin

Spray dried amorphous atorvastatin, an example of disordered atorvastatin as previously described in the Detailed Description of the Invention, and used in the following examples was prepared according to concurrently filed U.S. patent application, commonly owned, attorney case number PC-25825, Ser. No. ______, by first dissolving atorvastatin calcium (U.S. Pat. No. 5,273,995) in methanol to make a 5% (w:w) solution. This solution was sprayed into a Niro PSD-1 spray dryer at a rate of 170 g/min using nitrogen as the atomizing gas. The inlet temperature was 195° C. and the outlet temperature was 60° C. After spray drying, the powder was tray-dried in an oven at 40° C. for 12 hrs.

### EXAMPLE 2

### Preparation of Amorphous Atorvastatin Tablets Using a Wet Granulation

The following materials were added to a 950-cc amber bottle: 2.59 g of spray dried amorphous atorvastatin prepared as described in Example 1, 78.00 g of microcrystalline cellulose (Avicel™ PH102, FMC Biopolymer, Philadelphia, Pa.), 101.41 g of lactose (hydrous, Foremost Farms USA, Rothschild, Wis.), 6.00 g of croscarmellose sodium (Ac-Di-Sol™ FMC Biopolymer, Philadelphia, Pa.), and 4.000 g of hydroxypropyl cellulose (Klucel™ EXF, Hercules Incorporated, Aqualon Division, Wilmington, Del.). The materials were bottle blended for 10 minutes using a Turbula™ mixer (Turbula Shaker Mixer, Willy A. Bachofen AG Maschinenfabrik, Basel, Switzerland) and then discharged and sieved through a 30 mesh screen to delump. The material was then put back into the bottle and Turbula™ mixed an additional 10 minutes. The bottle-blended material was added to a Procept Mi-Mi-Pro high shear wet granulator (Pro-CepT n.v., B-9060 Zelzate, Belgium) using a 1.7 L bowl. The materials were dry mixed for two minutes at a chopper speed of 1000 revolutions per minute (rpm) and an impeller speed of 400 rpm, then the impeller speed was increased to 600 rpm maintaining the chopper speed. At this point, 90 mL of water was added at a rate of 30 mL/min. in three separate additions (60 mL, 15 mL, 15 mL) over a total of 5.5 minutes wet mixing. A good granulation with a minimum of fines was formed. The material was discharged and wet sieved by hand through a #10 mesh sieve. The sieved material was dried by placing on a polyethylene lined tray in a Gruenberg™ forced hot air oven (Gruenberg Oven Co., Williamsport, Pa.) at 50° C. for 16 hrs. The dried material was then milled using a Fitzpatrick L1A mill (The Fitzpatrick Co., Elmhurst, Ill.) with a 0.040″ Conidur rasping screen at 500 rpm. To 175.0 g of the blend was added 5.469 g of Ac-Di-Sol™ and the mixture was bottle blended (950-cc amber bottle) using a Turbula™ mixer for 5 minutes Magnesium stearate (Mallinckrodt Inc., St. Louis, Mo.) 1.822 g was then added and the mixture Turbula™ blended an additional 3 min. to complete the formulation. Tablets (˜250) were prepared using an F-press (Manesty F-Press, Liverpool, United Kingdom) with 13/32″ standard round concave (SRC) tooling, with a target weight of 450 mg (+3%) and a target hardness of 12 kP (range 10-14 kp). A total of 12 tablets were set up in 30-cc high density polyethylene (HDPE) bottles sealed using heat induction seal (HIS) closures, sealed using a heat induction sealer (Enercon Industries Corp., Menomonee, Wis.). Samples were stored for 4 weeks at 40° C. and 75% relative humidity (RH). Samples were analyzed for the level of atorvastatin lactone by adding one tablet to 50 mL of 1:1 (v:v) of a 0.05M ammonium citrate buffer (pH 7.4):acetonitrile and shaking for 20 minutes The material was then filtered using a Gelman Acrodisc polytetrafluoroethylene membrane (0.45 μm pore size), and analyzed using high-pressure liquid chromatography (HPLC) (Phenomenex, Ultremex C18 column, 25.0 cm×4.6 mm, HPLC HP 1100 series, Agilent Corp., Wilmington, Del., 20 μl injection volume, flow of 1.5 ml/min; mobile phase of 53:27:20 (v:v:v) 0.05M ammonium citrate (pH 4.0):acetonitrile:tetrahydrofuran; detection at 244 nm). The lactone level was found to be 25.4% (based on a ratio of the lactone peak to the total peak areas of all peaks).

### EXAMPLE 3

### Preparation of Amorphous Atrovastatin Calcium Tablets Using a Dry Granulation

The following materials were added to 950-cc amber glass bottle: 2.59 g of amorphous atorvastatin calcium prepared as described in Example 1, 78.00 g microcrystalline cellulose (Avicel PH102™; FMC Corp., Philadelphia, Pa.), 101.41 g lactose, hydrous (REG 310; Foremost Farms USA, Rothschild, Wis.), 4.00 g hydroxypropyl cellulose (Klucel EXF™; Aqualon, Wilmington, Del.), 6.00 g croscarmellose sodium (Ac-Di-Sol™; FMC Corp., Philadelphia, Pa.), and 1.00 g magnesium stearate (Mallinckrodt Co., St. Louis, Mo.). The combination of the above ingredients was mixed using a Turbula™ blender (Glen Mills, Clifton, N.J.) for 10 minutes The blend was then passed through a stainless steel sieve (#30 mesh) to delump, after which an additional 10 minutes of mixing was performed. The blend was then dry granulated by slugging with 1″ flat-faced tooling using a single station Manesty F-Press (Manesty, Liverpool, UK) to 1.00 g compacts with a hardness of 3.5 kP (tablet hardness was tested using a Schleuniger Tablet Hardness Tester, Dr. Schleuniger Pharmatron AG, Solothurn, Switzerland). The compacts were milled using a Fitzpatrick L1A mill (Fitzpatrick Co., Elmhurst, Ill.) with a 0.040″ Conidur rasping plate at 500 rpm. The recovered millings were returned to a glass bottle, to which 6.00 g croscarmellose sodium was added, and the contents blended for 5 min. Lastly, 1.00 g magnesium stearate was added to the amber glass bottle and the contents blended using the Turbula for 3 minutes Tablets were made using a single station Manesty F-Press. A 13/32″ standard round concave (SRC) punch and die was used to produce tablets with weights of 450 mg each. The average tablet hardness was 13 kilo pascals (kP) with a range of 12-14 kP. The average tablet weight was 447.9 mg with an RSD of 0.7%. Tablets were packaged, stored and analyzed as described in Example 2 which showed the level of atorvastatin lactone to be 0.17% (based on area percent of lactone peak).

### EXAMPLE 4

### Preparation and Analysis of Amorphous Atorvastatin Calcium Plus Excipient Blends—5% Drug

To each of ten 60-cc amber bottles, was added 500 mg of amorphous atorvastatin prepared as described in Example 1 and 9.4 g of one of the following excipients:

(a) xylitol (C granular, Danisco Sweeteners, Thomson, Ill.);

(b) mannitol (Mannogem™ 2080 granular, SPI Polyols, New Castle, Del.);

(c) sucrose (compressible sugar, White Di-Pac™, Tate & Lyle Co. American Sugars Inc, Brooklyn, N.Y.);

(d) lactose (spray dried monohydrate, Foremost Farms, Rothschild, Wis.);

(e) lactose (anhydrous, direct tableting grade, Quest International, Flavors & Food Ingredients CCL, Norwich, N.Y.);

(f) lactose (Fast Flo™ 316, Foremost Farms, Rothschild, Wis.);

(g) microcrystalline cellulose (Avicel™ PH102, FMC Biopolymer, Philadelphia, Pa.);

(h) microcrystalline cellulose (Avicel™ PH105, FMC Biopolymer, Philadelphia, Pa.);

(i) microcrystalline cellulose (Avicel™ PH101, FMC Biopolymer, Philadelphia, Pa.);

(j) calcium phosphate dibasic anhydrous (A-Tab™, Rhodia, Chicago Heights, Ill.).

Each mixture was blended for 15 minutes using a Turbula™ Shaker-Mixer (Willy A. Bachofen AG Maschinenfabrik, Basel, Switzerland). To each bottle was then added 100 mg of magnesium stearate (vegetable sourced, Mallinckrodt Inc., St. Louis, Mo.), and the mixtures were Turbula-blended for another 5 minutes. Sieve stacks were prepared with (from top to bottom) five spacers, a 60-mesh sieve, a 200-mesh sieve and a pan in the bottom. A piece of 6″ weighing paper was placed between the fourth and fifth spacers.

Each blend was separately placed on the 60-mesh sieve, and the sieve-stacks were placed into a Sonic Sifter™ (Allen Bradley Sonic Sifter, Advantech Manufacturing, New Berlin, Wis.). The blends were sifted for 6 minutes with sift and pulse amplitudes of 6. Weights in each sieve section were determined, and potency analyses were conducted by extracting the samples with 1:1 (v:v) deionized water:acetonitrile and shaking for 30 minutes. The material was then filtered using a Gelman Acrodisc™ polytetrafluroroethylene membrane (0.45 μm pore size), and analyzed using a UV-Vis Spectrophotometer (Model 8453, Agilent Corp., Wilmington, Del.). An external standard curve was used to quantitate the atorvastatin content. The weights and extraction volumes for each sample are shown in Table 1. Results are reported in Table 4.

TABLE 1
Sample preparation conditions for HPLC analyses. Dilution
involves taking the initial solution formed by combining the
amount analyzed with the extraction volume, and diluting
by the indicated amount with 1:1 (v:v) acetonitrile:water.

| | Material | | Material | | | |
|---|---|---|---|---|---|---|
| | retained on 60 | | retained on 200 | | | |
| | mesh sieve | | mesh sieve | | Fines | |
| | Amount | Extraction | Amount | Extraction | Amount | Extraction |
| | analyzed | volume | analyzed | volume | analyzed | volume |
| Example | (g) | (mL) | (g) | (mL) | (g) | (mL) |
| 4a | 8.9 | 1000 | 0.6 | 500 | 0.3 | 500 (dilute |
| | | | | | | 5:1) |
| 4b | 3.9 | 2000 | 0.7 | 500 | 0.5 | 1000 (dilute |
| | | | | | | 5:1) |
| 4c | 5.0 | 500 | 4.2 | 500 | 0.5 | 1000 (dilute |
| | | | | | | 5:1) |
| 4d | 0.2 | 100 | 7.2 | 1000 | 2.1 | 1000 |
| 4e | 1.3 | 500 | 5.2 | 500 (dilute | 3.1 | 500 (dilute |
| | | | | 5:1) | | 4:1) |
| 4f | 0.038 | 25 | 7.2 | 500 (dilute | 2.3 | 1000 |
| | | | | 6.67:1) | | |
| 4g | 0.017 | 10 | 5.0 | 500 | 4.8 | 500 (dilute |
| | | | | | | 5:1) |
| 4h | 0.007 | 10 | 0.070 | 25 | 9.8 | 1000 (dilute |
| | | | | | | 5:1) |
| 4i | | | 1.1 | 1000 | 1.9 | 1000 |
| 4j | 0.2 | 50 | 7.8 | 500 (dilute | 1.8 | 1000 (dilute |
| | | | | 6.67:1) | | 4:1) |

### EXAMPLE 5

### Preparation and Analysis of Amorphous Atorvastatin Calcium Plus Excipient Blends—40% Drug

To each of ten 60-cc amber bottles, was added 4.0 g of amorphous atorvastatin prepared as described in Example 1 and 5.8 g of one of the following excipients:

(a) xylitol (C granular, Danisco Sweeteners, Thomson, Ill.);

(b) mannitol (Mannogem™ 2080 granular, SPI Polyols, New Castle, Del.);

(c) sucrose (compressible sugar, White Di-Pac™, Tate & Lyle Co. American Sugars Inc, Brooklyn, N.Y.);

(d) lactose (spray dried monohydrate, Foremost Farms, Rothschild, Wis.);

(e) lactose (anhydrous, direct tableting grade, Quest International, Flavors & Food Ingredients CCL, Norwich, N.Y.);

(f) lactose (Fast Flo™ 316, Foremost Farms, Rothschild, Wis.);

(g) microcrystalline cellulose (Avicel™ PH102, FMC Biopolymer, Philadelphia, Pa.);

(h) microcrystalline cellulose (Avicel™ PH105, FMC Biopolymer, Philadelphia, Pa.);

(i) microcrystalline cellulose (Avicel™ PH101, FMC Biopolymer, Philadelphia, Pa.);

(j) calcium phosphate dibasic anhydrous (A-Tab™, Rhodia, Chicago Heights, Ill.).

Each mixture was blended for 15 minutes using a Turbula™ Shaker-Mixer (Willy A. Bachofen AG Maschinenfabrik, Basel, Switzerland). To each bottle was then added 200 mg of magnesium stearate (vegetable sourced, Mallinckrodt Inc., St. Louis, Mo.), and the mixtures were Turbula-blended for another 5 minutes. Sieve and potency analyses were conducted as described in Example 4, with extraction volumes reported in Table 2. Results of the analysis are reported in Table 4.

TABLE 2
Sample preparation conditions for HPLC analyses. Dilution
involves taking the initial solution formed by combining the
amount analyzed with the extraction volume, and diluting
by the indicated amount with 1:1 (v:v) acetonitrile:water.

| | Material | | Material | | | |
|---|---|---|---|---|---|---|
| | retained on 60 | | retained on 200 | | | |
| | mesh sieve | | mesh sieve | | Fines | |
| | Amount | Extraction | Amount | Extraction | Amount | Extraction |
| | analyzed | volume | analyzed | volume | analyzed | volume |
| Example | (g) | (mL) | (g) | (mL) | (g) | (mL) |
| 5a | 5.7 | 1000 | 0.2 | 500 | 1.9 | 1000 (dilute |
| | | | | | | 20:1) |
| 5b | 1.7 | 1000 | 0.9 | 500 | 0.9 | 1000 (dilute |
| | | | | | | 10:1) |
| 5c | 3.3 | 500 | 2.5 | 500 | 2.0 | 1000 (dilute |
| | | | | | | 20:1) |
| 5d | 0.1 | 500 | 2.8 | 1000 (dilute | 1.8 | 1000 (dilute |
| | | | | 10:1) | | 20:1) |
| 5e | 1.1 | 500 | 3.5 | 1000 (dilute | 2.5 | 1000 (dilute |
| | | | | 14.3:1) | | 20:1) |
| 5f | 0.024 | 100 | 3.4 | 1000 (dilute | 2.6 | 1000 (dilute |
| | | | | 20:1) | | 10:1) |
| 5g | 0.034 | 10 (dilute | 5.0 | 1000 (dilute | 2.2 | 1000 (dilute |
| | | 10:1) | | 20:1) | | 10:1) |
| 5h | 0.012 | 10 (dilute | 0.018 | 10 (dilute | 4.9 | 1000 (dilute |
| | | 10:1) | | 25:1) | | 20:1) |
| 5i | | | 1.5 | 1000 (dilute | 1.6 | 1000 (dilute |
| | | | | 5:1) | | 5:1) |
| 5j | 0.2 | 500 | 4.9 | 1000 (dilute | 4.7 | 1000 (dilute |
| | | | | 16.7:1) | | 20:1) |

### EXAMPLE 6

### Preparation and Analysis of Dry Granulations of Amorphous Atorvastatin Calcium Plus Excipient Blends

For the dry granulation process, true density was measured for each excipient and the atorvastatin at 25.1° C.±0.9° using a Micro-Ultrapycnometer 1000 (Quantachrome Corp., Boynton Beach, Fla.) with ultra high purity helium at 20 psig inlet pressure. All density measurements were performed using the large cell (cup volume 4.5 cm³) with the instrument programmed to operate in multi-run mode (maximum runs 15, runs to average 3, deviation 0.1%, purge mode flow, purge time 15 minutes). Reported values were from one replicate or the average of two replicates with new sample for each replicate. Sample weights were at least one gram (weight range 1.1 to 2.7 grams).

(a) The true density of a 5% (w:w) atorvastatin blend with xylitol was assumed to be a weighted average of the true densities of xylitol (1.49 g/cc) and atorvastatin prepared as described in Example 1; 1.24 g/cc, i.e., 1.48 g/cc. Compacts were made from a blend prepared as described in Example 4a using an F-press with rectangular tooling of 10×22 mm. The thicknesses and weights were varied to achieve a range of densities and corresponding solid fractions (i.e., density of the compact divided by 1.48 g/cc), in this case, the thicknesses, weights and solid fractions were 1.90 mm, 500 mg, 0.80; 2.16 mm, 597 mg, 0.84; 2.05 mm, 599 mg, 0.89; and 2.01 mm, 606 mg, 0.92. The corresponding deformation forces for the compacts were measured using a CT5 tensile strength Tester (Engineering System (NOTTM), Nottingham, England) and found to be 0.034, 0.042, 0.152 and 0.163 kg. These values were converted to tensile strengths by dividing the deformation force by the square of the thickness (multiplied by 22.07 to give the units in megapascals, MPa), corresponding to the following values: 0.21, 0.20, 0.80 and 0.89 MPa. Since this sample could not achieve the desired 1.0 MPa tensile strength, a maximal solid fraction of 0.92 was used for preparation of compacts. Based on this, compacts of the blend were prepared using 0.50″ round, flat-faced tooling on an F-press with 351 mg per compact and a thickness of 2.0 mm. These compacts (10 g total) were milled using a Mini Comil 193 (Quadro Engineering Incorporated, Waterloo, Ontario, Canada) with 0.040″ rasping screen, run at 900 rpm. Samples of material were analyzed as described in Example 4 with extraction volumes reported in Table 3, and analytical results reported in Table 4.

(b) The true density of a 5% (w:w) atorvastatin blend with mannitol was assumed to be a weighted average of the true densities of mannitol (1.45 g/cc) and atorvastatin prepared as described in Example 1; 1.24 g/cc, i.e., 1.44 g/cc. Compacts were prepared from a blend prepared as described in Example 4b using an F-press with rectangular tooling of 10×22 mm. The thicknesses, weights and solid fractions for compacts prepared as described in Example 6a were 1.98 mm, 460 mg, 0.73; 1.77 mm, 422 mg, 0.75; 1.61 mm, 394 mg, 0.77; 1.43 mm, 386 mg, 0.84; 2.07 mm, 552 mg, 0.84; 2.14 mm, 532 mg, 0.78; and 2.13 mm, 596 mg, 0.88. The corresponding tensile strengths for the compacts were 0.19, 0.25, 0.39, 1.03, 0.98, 0.40 and 1.92 MPa. The best-fit solid fraction for a 1.0 MPa tensile strength was found to be 0.84. Based on this, compacts were prepared as described in Example 6a with 305 mg per compact and a thickness of 1.95 mm. In addition, compacts were prepared with a tensile strength of 3.30 MPa, using 320 mg/compact at 1.90 mm thick. Both compacts were milled as described in Example 6a. Both samples of material were analyzed for particle size distribution by sieve analysis as described in Example 4. The latter sample was analyzed for potency as described in Example 4 with extraction volumes reported in Table 3 and the final results reported in Table 4.

(c) The true density of a 5% (w:w) atorvastatin blend with sucrose, direct tableting grade was assumed to be a weighted average of the true densities of the sucrose (1.52 g/cc) and atorvastatin prepared as described in Example 1; 1.24 g/cc, i.e., 1.51 g/cc. Compacts were prepared from a blend prepared as described in Example 4c using an F-press with rectangular tooling of 10×22 mm. The thicknesses, weights and solid fractions for compacts prepared as described in Example 6a were 1.56 mm, 397 mg, 0.76; 1.43 mm, 398 mg, 0.83; 2.10 mm, 604 mg, 0.86; 2.14 mm, 500 mg, 0.70; and 1.83 mm, 498 mg, 0.81. The corresponding tensile strengths for the compacts were 0.76, 2.06, 2.15, 0.34 and 1.15 MPa. The best-fit solid fraction for a 1.0 MPa tensile strength was found to be 0.78. Based on this, compacts of the blend (311 mg/compact, 2.00 mm thick) were prepared and milled as described in Example 6a. Samples of material were analyzed as described in Example 4 with extraction volumes reported in Table 3 and the final results reported in Table 4.

(d) The true density of a 5% (w:w) atorvastatin blend with lactose monohydrate was assumed to be a weighted average of the true densities of the lactose (1.49 g/cc) and atorvastatin prepared as described in Example 1; 1.24 g/cc, i.e., 1.48 g/cc. Compacts were prepared from a blend prepared as described in Example 4d using an F-press with rectangular tooling of 10×22 mm. The thicknesses, weights and solid fractions for compacts prepared as described in Example 6a were 1.54 mm, 415 mg, 0.81; 1.72 mm, 456 mg, 0.81; 1.92 mm, 478 mg, 0.76; 1.76 mm, 395 mg, 0.68; 1.98 mm, 488 mg, 0.75; and 1.84 mm, 506 mg, 0.83. The corresponding tensile strengths for the compacts were 1.55, 1.05, 0.74, 0.35, 0.60 and 1.83 MPa. The best-fit solid fraction for a 1.0 MPa tensile strength was found to be 0.78. Based on this, compacts of the blend (302 mg/compact, 2.02 mm thick) were prepared and milled as described in Example 6a. Samples of material were analyzed as described in Example 4 with extraction volumes reported in Table 3 and the final results reported in Table 4.

(e) The true density of a 5% (w:w) atorvastatin blend with lactose anhydrous was assumed to be a weighted average of the true densities of the lactose (1.50 g/cc) and atorvastatin prepared as described in Example 1; 1.24 g/cc, i.e., 1.49 g/cc. Compacts were prepared from a blend prepared as described in Example 4e using an F-press with rectangular tooling of 10×22 mm. The thicknesses, weights and solid fractions for compacts prepared as described in Example 6a were 1.82 mm, 427 mg, 0.71; 1.66 mm, 440 mg, 0.80; 1.58 mm, 430 mg, 0.82; and 1.82 mm, 479 mg, 0.80. The corresponding tensile strengths for the compacts were 0.68, 2.16, 2.52 and 1.80 MPa. The best-fit solid fraction for a 1.0 MPa tensile strength was found to be 0.75. Based on this, compacts of the blend (286 mg/compact, 2.02 mm thick) were prepared and milled as described in Example 6a. Samples of material were analyzed as described in Example 4 with extraction volumes reported in Table 3 and the final results reported in Table 4.

(f) The true density of a 5% (w:w) atorvastatin blend with lactose Fast Flo™ was assumed to be a weighted average of the true densities of the lactose (1.54 g/cc) and atorvastatin prepared as described in Example 1; 1.24 g/cc, i.e., 1.53 g/cc. Compacts were prepared from a blend prepared as described in Example 4f using an F-press with rectangular tooling of 10×22 mm. The thicknesses, weights and solid fractions for compacts prepared as described in Example 6a were 1.98 mm, 476 mg, 0.70; 1.80 mm, 440 mg, 0.72; 1.72 mm, 411 mg, 0.70; 1.80 mm, 342 mg, 0.55; and 1.73 mm, 475 mg, 0.80. The corresponding tensile strengths for the compacts were 1.10, 1.27, 1.16, 0.13 and 2.61 MPa. The best-fit solid fraction for a 1.0 MPa tensile strength was found to be 0.70. Based on this, compacts of the blend (272 mg/compact, 2.01 mm thick) were prepared and milled as described in Example 6a. Samples of material were analyzed as described in Example 4 with extraction volumes reported in Table 3 and the final results reported in Table 4.

(g) The true density of a 5% (w:w) atorvastatin blend with microcrystalline cellulose (Avicel™ PH102) was assumed to be a weighted average of the true densities of the microcrystalline cellulose (1.58 g/cc) and atorvastatin prepared as described in Example 1; 1.24 g/cc, i.e., 1.56 g/cc. Compacts were prepared from a blend prepared as described in Example 4g using an F-press with rectangular tooling of 10×22 mm. The thicknesses, weights and solid fractions for compacts prepared as described in Example 6a were 2.56 mm, 417 mg, 0.47; 1.83 mm, 418 mg, 0.66; 1.60 mm, 420 mg, 0.76; 2.29 mm, 382 mg, 0.48; 1.70 mm, 383 mg, 0.65; and 1.91 mm, 347 mg, 0.52. The corresponding tensile strengths for the compacts were 0.22, 3.36, 6.99, 0.64, 2.58 and 1.03 MPa. The best-fit solid fraction for a 1.0 MPa tensile strength was found to be 0.56. Based on this, compacts of the blend (226 mg/compact, 2.01 mm thick) were prepared and milled as described in Example 6a. Samples of material were analyzed as described in Example 4 with extraction volumes reported in Table 3 and the final results reported in Table 4.

(h) The true density of a 5% (w:w) atorvastatin blend with microcrystalline cellulose (Avicel™ PH105) was assumed to be a weighted average of the true densities of the microcrystalline cellulose (1.55 g/cc) and atorvastatin prepared as described in Example 1; 1.24 g/cc, i.e., 1.53 g/cc. Compacts were prepared from a blend prepared as described in Example 4h using an F-press with rectangular tooling of 10×22 mm. The thicknesses, weights and solid fractions for compacts prepared as described in Example 6a were 1.87 mm, 432 mg, 0.68; 1.55 mm, 387 mg, 0.73; 2.21 mm, 390 mg, 0.52; 1.63 mm, 329 mg, 0.59; 2.18 mm, 311 mg, 0.42; and 1.35 mm, 258 mg, 0.56. The corresponding tensile strengths for the compacts were 4.17, 7.99, 1.29, 2.62, 0.27 and 2.31 MPa. The best-fit solid fraction for a 1.0 MPa tensile strength was found to be 0.51. Based on this, compacts of the blend (211 mg/compact, 2.03 mm thick) were prepared and milled as described in Example 6a. Samples of material were analyzed as described in Example 4 with extraction volumes reported in Table 3 and the final results reported in Table 4.

(i) The true density of a 5% (w:w) atorvastatin blend with microcrystalline cellulose (Avicel™ PH101) was assumed to be a weighted average of the true densities of the microcrystalline cellulose (1.56 g/cc) and atorvastatin prepared as described in Example 1; 1.24 g/cc, i.e., 1.54 g/cc. Compacts were prepared from a blend prepared as described in Example 4i using an F-press with rectangular tooling of 10×22 mm. The thicknesses, weights and solid fractions for compacts prepared as described in Example 6a were 2.06 mm, 541 mg, 0.77; 2.09 mm, 506 mg, 0.71; 1.96 mm, 478 mg, 0.71; 2.20 mm, 432 mg, 0.57; and 1.84 mm, 450 mg, 0.72. The corresponding tensile strengths for the compacts were 7.25, 4.96, 5.11, 1.79, and 5.11 MPa. The best-fit solid fraction for a 1.0 MPa tensile strength was extrapolated to be 0.50. Based on this, compacts of the blend (208 mg/compact, 2.06 mm thick) were prepared and milled as described in Example 6a. Samples of material were analyzed as described in Example 4 with extraction volumes reported in Table 3 and the final results reported in Table 4.

(j) The true density of a 5% (w:w) atorvastatin blend with calcium phosphate dibasic anhydrous (A-Tab™) was assumed to be a weighted average of the true densities of the calcium phosphate (2.78 g/cc) and atorvastatin prepared as described in Example 1; 1.24 g/cc, i.e., 2.70 g/cc. Compacts were prepared from a blend prepared as described in Example 4j using an F-press with rectangular tooling of 10×22 mm. The thicknesses, weights and solid fractions for compacts prepared as described in Example 6a were 2.22 mm, 706 mg, 0.53; 1.82 mm, 598 mg, 0.55; 2.29 mm, 796 mg, 0.58; and 2.05 mm, 598 mg, 0.49. The corresponding tensile strengths for the compacts were 0.98, 1.55, 2.32 and 0.49 MPa. The best-fit solid fraction for a 1.0 MPa tensile strength was found to be 0.53. Based on this, compacts of the blend (357 mg/compact, 1.94 mm thick) were prepared and milled as described in Example 6a. Samples of material were analyzed as described in Example 4 with extraction volumes reported in Table 3 and the final results reported in Table 4.

(k) The true density of a 40% (w:w) atorvastatin blend with xylitol was assumed to be a weighted average of the true densities of xylitol (1.49 g/cc) and atorvastatin prepared as described in Example 1; 1.24 g/cc, i.e., 1.39 g/cc. Compacts were prepared from a blend prepared as described in Example 5a using an F-press with rectangular tooling of 10×22 mm. The thicknesses, weights and solid fractions for compacts prepared as described in Example 6a were 1.66 mm, 434 mg, 0.84; 2.03 mm, 535 mg, 0.85; 1.95 mm, 530 mg, 0.88; 2.00 mm, 431 mg, 0.69; 2.14 mm, 587 mg, 0.88; and 2.28 mm, 595 mg, 0.84. The corresponding tensile strengths for the compacts were 0.62, 0.98, 1.19, 0.09, 1.31 and 0.71 MPa. The best-fit solid fraction for a 1.0 MPa tensile strength was found to be 0.86. Based on this, compacts of the blend (296 mg/compact, 1.92 mm thick) were prepared and milled as described in Example 6a. Samples of material were analyzed as described in Example 4 with extraction volumes reported in Table 3 and the final results reported in Table 5.

(l) The true density of a 40% (w:w) atorvastatin blend with mannitol was assumed to be a weighted average of the true densities of mannitol (1.45 g/cc) and atorvastatin prepared as described in Example 1; 1.24 g/cc, i.e., 1.37 g/cc. Compacts were prepared from a blend prepared as described in Example 5b using an F-press with rectangular tooling of 10×22 mm. The thicknesses, weights and solid fractions for compacts prepared as described in Example 6a were 1.97 mm, 426 mg, 0.71; 1.97 mm, 455 mg, 0.76; 1.79 mm, 460 mg, 0.84; 1.97 mm, 485 mg, 0.81; 1.90 mm, 519 mg, 0.90; and 1.93 mm, 516 mg, 0.88. The corresponding tensile strengths for the compacts were 0.63, 0.84, 2.13, 1.74, 2.91 and 2.72 MPa. The best-fit solid fraction for a 1.0 MPa tensile strength was found to be 0.76. Based on this, compacts were prepared as described in Example 6a with 269 mg per compact and a thickness of 2.00 mm. In addition, compacts were prepared with a tensile strength of 2.18 MPa, using 300 mg/compact at 1.98 mm thick. Both compacts were milled as described in Example 6a. Both samples of material were analyzed for particle size distribution by sieve analysis as described in Example 4. The latter sample was analyzed for potency as described in Example 4 with extraction volumes reported in Table 3 and the final results reported in Table 5.

(m) The true density of a 40% (w:w) atorvastatin blend with sucrose, direct tableting grade, was assumed to be a weighted average of the true densities of the sucrose (1.52 g/cc) and atorvastatin prepared as described in Example 1; 1.24 g/cc, i.e., 1.41 g/cc. Compacts were prepared from a blend prepared as described in Example 5c using an F-press with rectangular tooling of 10×22 mm. The thicknesses, weights and solid fractions for compacts prepared as described in Example 6a were 2.08 mm, 403 mg, 0.62; 2.00 mm, 466 mg, 0.74; 1.66 mm, 412 mg, 0.79; 1.73 mm, 467 mg, 0.86; 2.12 mm, 478 mg, 0.72; 1.82 mm, 481 mg, 0.84; and 1.83 mm, 478 mg, 0.83. The corresponding tensile strengths for the compacts were 0.20, 0.74, 1.43, 2.07, 0.46, 2.31, and 1.98 MPa. The best-fit solid fraction for a 1.0 MPa tensile strength was found to be 0.77. Based on this, compacts of the blend (298 mg/compact, 2.13 mm thick) were prepared and milled as described in Example 6a. Samples of material were analyzed as described in Example 4 with extraction volumes reported in Table 3 and the final results reported in Table 5.

(n) The true density of a 40% (w:w) atorvastatin blend with lactose monohydrate was assumed to be a weighted average of the true densities of the lactose (1.49 g/cc) and atorvastatin prepared as described in Example 1; 1.24 g/cc, i.e., 1.39 g/cc. Compacts were prepared from a blend prepared as described in Example 5d using an F-press with rectangular tooling of 10×22 mm. The thicknesses, weights and solid fractions for compacts prepared as described in Example 6a were 2.09 mm, 541 mg, 0.83; 1.90 mm, 471 mg, 0.80; 1.54 mm, 331 mg, 0.69; and 2.18 mm, 594 mg, 0.88. The corresponding tensile strengths for the compacts were 1.79, 1.34, 0.73, and 2.62 MPa. The best-fit solid fraction for a 1.0 MPa tensile strength was found to be 0.74. Based on this, compacts of the blend (265 mg/compact, 1.96 mm thick) were prepared and milled as described in Example 6a. Samples of material were analyzed as described in Example 4 with extraction volumes reported in Table 3 and the final results reported in Table 5.

(o) The true density of a 40% (w:w) atorvastatin blend with lactose anhydrous was assumed to be a weighted average of the true densities of the lactose (1.50 g/cc) and atorvastatin prepared as described in Example 1; 1.24 g/cc, i.e., 1.40 g/cc. Compacts were prepared from a blend prepared as described in Example 5e using an F-press with rectangular tooling of 10×22 mm. The thicknesses, weights and solid fractions for compacts prepared as described in Example 6a were 1.85 mm, 400 mg, 0.69; 1.97 mm, 467 mg, 0.76; 2.07 mm, 501 mg, 0.77; 2.01 mm, 527 mg, 0.84; and 2.00 mm, 398 mg, 0.64. The corresponding tensile strengths for the compacts were 0.81, 1.58, 1.52, 2.80, and 0.37 MPa. The best-fit solid fraction for a 1.0 MPa tensile strength was found to be 0.72. Based on this, compacts of the blend (278 mg/compact, 2.08 mm thick) were prepared and milled as described in Example 6a. Samples of material were analyzed as described in Example 4 with extraction volumes reported in Table 3 and the final results reported in Table 5.

(p) The true density of a 40% (w:w) atorvastatin blend with lactose (FastFlo™) was assumed to be a weighted average of the true densities of the lactose (1.54 g/cc) and atorvastatin prepared as described in Example 1; 1.24 g/cc, i.e., 1.42 g/cc. Compacts were prepared from a blend prepared as described in Example 5f using an F-press with rectangular tooling of 10×22 mm. The thicknesses, weights and solid fractions for compacts prepared as described in Example 6a were 2.26 mm, 360 mg, 0.50; 1.97 mm, 375 mg, 0.60; 2.14 mm, 409 mg, 0.60; 1.92 mm, 437 mg, 0.72; and 2.17 mm, 530 mg, 0.77. The corresponding tensile strengths for the compacts were 0.09, 0.44, 0.43, 1.15 and 2.02 MPa. The best-fit solid fraction for a 1.0 MPa tensile strength was found to be 0.70. Based on this, compacts of the blend (262 mg/compact, 1.99 mm thick) were prepared and milled as described in Example 6a. Samples of material were analyzed as described in Example 4 with extraction volumes reported in Table 3 and the final results reported in Table 5.

(q) The true density of a 40% (w:w) atorvastatin blend with microcrystalline cellulose (Avicel™ PH102) was assumed to be a weighted average of the true densities of the microcrystalline cellulose (1.58 g/cc) and atorvastatin prepared as described in Example 1; 1.24 g/cc, i.e., 1.44 g/cc. Compacts were prepared from a blend prepared as described in Example 5g using an F-press with rectangular tooling of 10×22 mm. The thicknesses, weights and solid fractions for compacts prepared as described in Example 6a were 1.90 mm, 343 mg, 0.56; 1.88 mm, 400 mg, 0.66; 2.10 mm, 441 mg, 0.65; and 2.30 mm, 366 mg, 0.49. The corresponding tensile strengths for the compacts were 0.78, 2.28, 1.88, and 0.29 MPa. The best-fit solid fraction for a 1.0 MPa tensile strength was found to be 0.59. Based on this, compacts of the blend (223 mg/compact, 2.04 mm thick) were prepared and milled as described in Example 6a. Samples of material were analyzed as described in Example 4 with extraction volumes reported in Table 3 and the final results reported in Table 5.

(r) The true density of a 40% (w:w) atorvastatin blend with microcrystalline cellulose (Avicel™ PH105) was assumed to be a weighted average of the true densities of the microcrystalline cellulose (1.55 g/cc) and atorvastatin prepared as described in Example 1; 1.24 g/cc, i.e., 1.43 g/cc. Compacts were prepared from a blend prepared as described in Example 5h using an F-press with rectangular tooling of 10×22 mm. The thicknesses, weights and solid fractions for compacts prepared as described in Example 6a were 1.91 mm, 335 mg, 0.55; 1.82 mm, 312 mg, 0.54; 1.90 mm, 399 mg, 0.66; 2.23 mm, 393 mg, 0.55; 2.10 mm, 445 mg, 0.67; and 1.82 mm, 433 mg, 0.75. The corresponding tensile strengths for the compacts were 0.85, 0.66, 2.22, 0.76, 2.00, and 4.42 MPa. The best-fit solid fraction for a 1.0 MPa tensile strength was found to be 0.58. Based on this, compacts of the blend (228 mg/compact, 2.03 mm thick) were prepared and milled as described in Example 6a. Samples of material were analyzed as described in Example 4 with extraction volumes reported in Table 3 and the final results reported in Table 5.

(s) The true density of a 40% (w:w) atorvastatin blend with microcrystalline cellulose (Avicel™ PH101) was assumed to be a weighted average of the true densities of the microcrystalline cellulose (1.56 g/cc) and atorvastatin prepared as described in Example 1; 1.24 g/cc, i.e., 1.43 g/cc. Compacts were prepared from a blend prepared as described in Example 5i using an F-press with rectangular tooling of 10×22 mm. The thicknesses, weights and solid fractions for compacts prepared as described in Example 6a were 1.59 mm, 368 mg, 0.73; 2.24 mm, 397 mg, 0.56; 1.97 mm, 460 mg, 0.73; 1.81 mm, 363 mg, 0.63; 1.69 mm, 394 mg, 0.73; and 1.97 mm, 381 mg, 0.61. The corresponding tensile strengths for the compacts were 3.66, 0.75, 3.90, 1.62, 3.72, and 1.50 MPa. The best-fit solid fraction for a 1.0 MPa tensile strength was found to be 0.58. Based on this, compacts of the blend (209 mg/compact, 2.00 mm thick) were prepared and milled as described in Example 6a. Samples of material were analyzed as described in Example 4 with extraction volumes reported in Table 3 and the final results reported in Table 5.

(t) The true density of a 40% (w:w) atorvastatin blend with calcium phosphate dibasic anhydrous (A-Tab™) was assumed to be a weighted average of the true densities of the calcium phosphate (2.78 g/cc) and atorvastatin prepared as described in Example 1; 1.24 g/cc, i.e., 2.16 g/cc. Compacts were prepared from a blend prepared as described in Example 5j using an F-press with rectangular tooling of 10×22 mm. The thicknesses, weights and solid fractions for compacts prepared as described in Example 6a were 1.92 mm, 525 mg, 0.57; 1.79 mm, 484 mg, 0.56; 1.87 mm, 485 mg, 0.54; 1.99 mm, 579 mg, 0.60; and 2.10 mm, 481 mg, 0.48. The corresponding tensile strengths for the compacts were 1.56, 1.31, 1.13, 1.95, and 0.45 MPa. The best-fit solid fraction for a 1.0 MPa tensile strength was found to be 0.54. Based on this, compacts of the blend (294 mg/compact, 1.97 mm thick) were prepared and milled as described in Example 4 with extraction volumes reported in Table 3 and the final results reported in Table 5.

TABLE 3
Sample preparation conditions for HPLC analyses. Dilution
involves taking the initial solution formed by combining the
amount analyzed with the extraction volume, and diluting
by the indicated amount with 1:1 (v:v) acetonitrile:water.

| | Material | | Material | | | |
|---|---|---|---|---|---|---|
| | retained on 60 | | retained on 200 | | | |
| | mesh sieve | | mesh sieve | | Fines | |
| | Amount | Extraction | Amount | Extraction | Amount | Extraction |
| | analyzed | volume | analyzed | volume | analyzed | volume |
| Example | (g) | (mL) | (g) | (mL) | (g) | (mL) |
| 6a | 4.9 | 500 (dilute | 2.8 | 1000 | 1.7 | 1000 |
| | | 5:1) | | | | |
| 6b | 1.5 | 1000 | 1.4 | 1000 | 0.5 | 1000 |
| 6c | 4.3 | 500 (dilute | 2.9 | 500 (dilute | 1.8 | 1000 |
| | | 4:1) | | 4:1) | | |
| 6d | 3.5 | 1000 | 3.0 | 1000 | 2.4 | 1000 |
| 6e | 4.0 | 500 (dilute | 3.1 | 1000 | 2.2 | 1000 |
| | | 4:1) | | | | |
| 6f | 4.6 | 500 (dilute | 1.5 | 500 | 3.3 | 1000 |
| | | 4:1) | | | | |
| 6g | 2.3 | 1000 | 4.0 | 500 (dilute | 3.0 | 1000 |
| | | | | 5:1) | | |
| 6h | 4.9 | 500 (dilute | 1.2 | 500 | 3.5 | 500 (dilute |
| | | 5:1) | | | | 5:1) |
| 6i | 2.2 | 1000 | 2.3 | 1000 | 5.0 | 500 (dilute |
| | | | | | | 5:1) |
| 6j | 4.2 | 500 (dilute | 3.2 | 500 (dilute | 2.4 | 1000 |
| | | 4:1) | | 4:1) | | |
| 6k | 3.2 | 1000 (dilute | 1.8 | 500 (dilute | 1.0 | 500 (dilute |
| | | 10:1) | | 14.3:1) | | 10:1) |
| 6l | 1.7 | 1000 (dilute | 1.5 | 1000 (dilute | 0.5 | 1000 (dilute |
| | | 5:1) | | 5:1) | | 4:1) |
| 6m | 4.9 | 1000 (dilute | 2.5 | 1000 (dilute | 1.8 | 1000 |
| | | 20:1) | | 10:1) | | (6.67:1) |
| 6n | 4.9 | 1000 (dilute | 2.6 | 1000 (dilute | 1.5 | 1000 (dilute |
| | | 20:1) | | 10:1) | | 6.67:1) |
| 6o | 2.8 | 1000 (dilute | 2.3 | 1000 (dilute | 1.3 | 500 (dilute |
| | | 10:1) | | 10:1) | | 10:1) |
| 6p | 3.0 | 1000 (dilute | 1.5 | 500 (dilute | 1.7 | 500 (dilute |
| | | 10:1) | | 10:1) | | 10:1) |
| 6q | 2.2 | 1000 (dilute | 3.1 | 1000 (dilute | 1.7 | 1000 (dilute |
| | | 10:1) | | 10:1) | | 10:1) |
| 6r | 2.5 | 1000 (dilute | 1.0 | 500 (dilute | 3.0 | 1000 (dilute |
| | | 10:1) | | 10:1) | | 10:1) |
| 6s | 2.2 | 1000 (dilute | 2.2 | 500 (dilute | 2.2 | 1000 (dilute |
| | | 10:1) | | 10:1) | | 10:1) |
| 6t | 2.5 | 1000 (dilute | 2.4 | 500 (dilute | 2.0 | 500 (dilute |
| | | 10:1) | | 20:1) | | 20:1) |

TABLE 4
Comparison of simple blends versus dry granulations
with atorvastatin and a series of diluents (with atorvastatin
at 5 wt %) showing the beneficial effects on segregation
of certain diluents.

| | Material | | Material | | | | |
|---|---|---|---|---|---|---|---|
| | retained on | | retained on | | | | |
| | 60 mesh sieve | | 200 mesh sieve | | Fines | | |
| | | Potency | | Potency | | Potency | Granulation |
| Example | Weight (g) | (mgA/g) | Weight (g) | (mgA/g) | Weight (g) | (mgA/g) | Factor |
| 4a | 9.010 | 9.9 | 0.628 | 35.5 | 0.318 | 837.7 | 0.56 |
| 6a | 5.026 | 31.5 | 2.937 | 29.9 | 1.722 | 64.6 | |
| 4b | 8.003 | 3.9 | 1.406 | 7.3 | 0.557 | 782.6 | 0.74 |
| 6b | 4.788 | 39.0 | 1.549 | 37.8 | 1.413 | 54.2 | |
| 4c | 5.197 | 1.9 | 4.201 | 2.3 | 0.554 | 686.8 | 0.49 |
| 6c | 4.495 | 25.4 | 3.057 | 13.7 | 1.816 | 81.8 | |
| 4d | 0.221 | 7.3 | 7.613 | 21.1 | 2.149 | 78.7 | 0.23 |
| 6d | 3.622 | 40.9 | 3.162 | 21.3 | 2.394 | 58.5 | |
| 4e | 1.414 | 1.5 | 5.418 | 1.8 | 3.145 | 126.3 | 0.57 |
| 6e | 4.167 | 37.3 | 3.272 | 20.0 | 2.221 | 70.3 | |
| 4f | 0.081 | 38.5 | 7.593 | 31.7 | 2.281 | 67.2 | 0.06 |
| 6f | 4.651 | 44.8 | 1.653 | 32.7 | 3.308 | 45.0 | |
| 4g | 0.052 | 15.0 | 5.174 | 6.8 | 4.783 | 78.7 | 0.50 |
| 6g | 2.404 | 42.7 | 4.187 | 22.4 | 3.028 | 53.6 | |
| 4h | 0.048 | 44.1 | 0.119 | 40.3 | 9.814 | 47.4 | 0.65 |
| 6h | 4.937 | 47.2 | 1.214 | 35.2 | 3.491 | 42.2 | |
| 4i | 0.029 | — | 2.420 | 5.5 | 7.524 | 52.2 | 0.37 |
| 6i | 2.221 | 41.1 | 2.440 | 19.2 | 5.009 | 43.0 | |
| 4j | 0.206 | 8.8 | 7.910 | 6.2 | 1.860 | 204.6 | 0.53 |
| 6j | 4.214 | 41.5 | 3.231 | 24.9 | 2.390 | 75.6 | |

TABLE 5
Comparison of simple blends versus dry granulations
with atorvastatin and a series of diluents (with atorvastatin
at 40 wt %) showing the beneficial effects on segregation
of certain diluents.

| | Material | | Material | | | | |
|---|---|---|---|---|---|---|---|
| | retained on 60 | | retained on | | | | |
| | mesh sieve | | 200 mesh sieve | | Fines | | |
| | | Potency | | Potency | | Potency | Granulation |
| Example | Weight (g) | (mgA/g) | Weight (g) | (mgA/g) | Weight (g) | (mgA/g) | Factor |
| 5a | 5.881 | 24.1 | 0.248 | 101.8 | 3.799 | 893.2 | 0.84 |
| 6k | 6.462 | 325.7 | 1.930 | 333.4 | 0.970 | 522.9 | |
| 5b | 5.260 | 33.0 | 1.011 | 83.4 | 3.681 | 824.0 | 0.73 |
| 6l | 5.283 | 303.3 | 1.577 | 367.2 | 1.361 | 518.2 | |
| 5c | 3.367 | 13.3 | 2.541 | 8.4 | 4.005 | 881.9 | 0.62 |
| 6m | 5.063 | 284.0 | 2.624 | 233.7 | 1.851 | 672.7 | |
| 5d | 0.205 | 54.3 | 6.064 | 191.0 | 3.678 | 627.1 | 0.64 |
| 6n | 4.992 | 361.1 | 2.764 | 251.3 | 1.565 | 499.5 | |
| 5e | 1.129 | 29.1 | 3.869 | 66.8 | 4.955 | 633.0 | 0.79 |
| 6o | 5.807 | 348.5 | 2.454 | 292.7 | 1.319 | 490.6 | |
| 5f | 0.059 | 146.0 | 7.224 | 245.5 | 2.634 | 650.5 | 0.62 |
| 6p | 6.224 | 364.6 | 1.666 | 315.3 | 1.691 | 376.0 | |
| 5g | 0.112 | 114.7 | 5.413 | 184.0 | 4.426 | 558.8 | 0.24 |
| 6q | 2.295 | 359.1 | 3.425 | 180.4 | 3.506 | 483.3 | |
| 5h | 0.071 | 235.0 | 0.106 | 518.4 | 9.788 | 384.6 | 0.69 |
| 6r | 5.213 | 383.1 | 1.195 | 360.0 | 3.024 | 350.7 | |
| 5i | 0.087 | — | 4.920 | 269.2 | 4.917 | 397.8 | 0.02 |
| 6s | 2.263 | 351.4 | 2.502 | 202.9 | 4.575 | 404.9 | |
| 5j | 0.206 | 67.1 | 5.011 | 15.1 | 4.739 | 708.7 | 0.68 |
| 6t | 5.300 | 346.1 | 2.499 | 251.1 | 2.000 | 550.7 | |

What is claimed is:

1. A dry-granulated pharmaceutical composition comprising atorvastatin or a pharmaceutically acceptable salt thereof.

2. The pharmaceutical composition according to claim 1 wherein the composition contains less than about 5% (w:w) of an alkaline earth metal salt additive.

3. The pharmaceutical composition according to claim 1 wherein the dry-granulated composition, after storage at 40° C. and 75% relative humidity for 4 weeks, contains not more than about 2% total impurities and/or degradants based on area percent of drug related HPLC peaks.

4. The pharmaceutical composition according to claim 1 wherein the composition contains not more than about 2% atorvastatin lactone based on area percent of HPLC peaks.

5. The pharmaceutical composition according to claim 1 wherein the composition is used in the formation of a solid unit dosage form.

6. The pharmaceutical composition according to claim 5 wherein the unit dosage form is selected from the group consisting of a tablet and a capsule.

7. The pharmaceutical composition according to claim 1 wherein the atorvastatin contains at least some partially or completely disordered form of atorvastatin or a pharmaceutically acceptable salt thereof.

8. The pharmaceutical composition according to claim 5 wherein the unit dosage form, after storage at 40° C. and 75% relative humidity for 4 weeks, contains not more than about 1% total impurities and/or degradants based on area percent of drug related HPLC peaks.

9. The pharmaceutical composition according to claim 5 wherein the unit dosage form, after storage at 40° C. and 75% relative humidity for 4 weeks, contains not more than about 1% of atorvastatin lactone as calculated by area integration of HPLC peaks.

10. The pharmaceutical composition according to claim 1 wherein the composition comprises a diluent.

11. The pharmaceutical composition according to claim 10 wherein said diluent has a mean particle size between about 20 and 200 μm.

12. The pharmaceutical composition according to claim 10 wherein said diluent has a mean particle size between 40 and 150 μm.

13. The pharmaceutical composition according to claim 1 wherein said composition shows a granulation factor of between about 0.4 and 1.0.

14. The pharmaceutical composition according to claim 1 wherein said composition shows a granulation factor of between about 0.5 and 1.0.

15. The pharmaceutical composition according to claim 1 wherein said composition shows a granulation factor of between about 0.6 and 1.0.

16. The pharmaceutical composition of claim 1 wherein said composition comprises greater than 40% (w:w) of a diluent or combination of diluents wherein said diluent or combination of diluents have a granulation factor between 0.4 and 1.0 when tested alone with atorvastatin.

17. The pharmaceutical composition according to claim 12 wherein said diluent comprises greater than about 50% (w:w) of microcrystalline cellulose, lactose, sucrose, xylitol or calcium phosphate dibasic.

18. The unit dosage form according to claim 5 wherein said unit dosage form produced therein shows a relative standard deviation for active drug per unit dosage form of less than 7.8% when said unit dosage form is prepared at a rate greater than about 10,000 unit dosage forms per hour per unit dosage form per machine.

19. The unit dosage form according to claim 5 wherein said unit dosage form produced therein shows a relative standard deviation for active drug per unit dosage form of less than 6.0% when said unit dosage form is prepared at a rate greater than about 10,000 unit dosage forms per hour per unit dosage form per machine.

20. The unit dosage form according to claim 5 wherein said dosage form also contains at least one active drug in addition to the atovastatin.

21. The unit dosage form according to claim 20 wherein said active drug in addition to the atorvastatin includes torcetrapib or amlodipine and pharmaceutically acceptable salts thereof.

22. A method for preparing a dry-granulated pharmaceutical composition of atorvastatin comprising:
a. combining atorvastatin or a pharmaceutically acceptable salt thereof and one or more excipients suitable for use in a dry granulation step;
b. blending the mixture together in a mixer;
c. compressing the mixture;
d. milling, grinding or sieving the compressed material;
e. optionally adding additional excipients and mixing the combination to form the composition.

23. The method according to claim 22 wherein said compression is carried out using a roller compactor.

24. The method according to claim 22 wherein said compression is carried out using a tablet press.

25. The method according to claim 22 wherein said compression provides a material having a tensile strength of about 0.55 to about 8 MPa.

26. The method according to claim 22 wherein said compression provides a material having a tensile strength of about 0.8 to about 6 MPa.

27. The method according to claim 22 wherein said compression provides a material having a solid fraction of between about 0.55 and about 0.85.

28. The method according to claim 22 wherein said compression provides a material having a solid fraction of between about 0.60 and about 0.80.

29. The method according to claim 22 wherein said milling, grinding or sieving provides a material wherein less than about 30% (w:w) of the material passes through a 200 mesh sieve.

30. The method according to claim 22 wherein said milling, grinding or sieving provides a material wherein greater than about 70% (w:w) of the material passes through a 60 mesh sieve.

31. The method according to claim 22 wherein the material after milling, grinding, or sieving in step (d) provides a granulation factor of between about 0.4 and about 1.0.

32. The method according to claim 22 wherein the material after milling grinding, or sieving in step (d) provides a granulation factor of between about 0.5 and about 1.0.

33. The method according to claim 22 wherein the material after milling grinding, or sieving in step (d) provides a granulation factor of between about 0.6 and about 1.0.

34. The method according to claim 22 wherein the composition is used in the preparation of tablets or capsules.

35. The method according to claim 34 wherein said tablets or capsules produced therein show a relative standard deviation for active drug per unit dosage form of less than 7.8% when said unit dosage forms are prepared on a tablet press or capsule filling machine at a rate greater than about 10,000 tablets or capsules per hour per unit dosage form per machine.

36. The method according to claim 34 wherein said tablets or capsules produced therein show a relative standard deviation for active drug per unit dosage form of less than 6.0% when said unit dosage forms are prepared on a tablet press or capsule filling machine at a rate greater than 10,000 tablets or capsules per hour per unit dosage form per machine.

37. The method according to claim 22 wherein said atorvastatin comprises at least some amount of a partially or completely disordered form of atorvastatin or a pharmaceutically acceptable salt thereof.

38. The method of preparing a unit dosage form containing atorvastatin and at least one other active drug wherein the composition prepared according to the method of claim 22 is combined with at least one other active drug and optionally additional excipients.

39. The method of treating hypercholesterolemia and/or hyperlipidemia, osteoporosis, benign prostatic hyperplasia, and Alzheimer's disease comprising administering a therapeutically effective amount of the pharmaceutical composition of claim 1.

40. A kit for achieving a therapeutic effect in a mammal comprising a therapeutically effective amount of dry-granulated atorvastatin or a pharmaceutically acceptable salt thereof step in a unit dosage form, and a container for containing said dosage form.

41. The kit according to claim 39 containing at least some partially or completely disordered form of atorvastatin or a pharmaceutically acceptable salt thereof.

42. The kit according to claim 39 wherein the unit dosage form is selected from the group consisting of a tablet or a capsule.
